(19) United States
(12) Patent Application Publication
(10) Pub. No.: US 2020/0060323 A1
(43) Pub. Date: Feb. 27, 2020

(54) PGC-1 ALPHA PROTEIN EXPRESSION PROMOTER AND SLOW-TO-FAST MUSCLE CONVERSION INHIBITOR

(71) Applicants: Kaneka Corporation, Osaka-shi (JP); National University Corporation Kobe University, Kobe-shi (JP)

(72) Inventors: Hidemi FUJINO, Kobe-shi (JP); Airo TATEGAKI, Takasago-shi (JP)

(73) Assignees: KANEKA CORPORATION, Osaka-shi (JP); NATIONAL UNIVERSITY CORPORATION KOBE UNIVERSITY, Kobe-shi (JP)

(21) Appl. No.: 16/669,838

(22) Filed: Oct. 31, 2019

Related U.S. Application Data
(63) Continuation of application No. PCT/JP2018/017661, filed on May 7, 2018.

(30) Foreign Application Priority Data
May 8, 2017 (JP) 2017-092263

(51) Int. Cl. A23L 33/135; A61K 9/00; A61K 35/744

(52) U.S. Cl. CPC A23L 33/135; A23L 2/60; A61K 35/744; A61K 9/0053

(57) ABSTRACT

A food or a drink, including: Enterococcus faecium R30 strain (NITE BP-01362) or a processed product thereof; and at least one additive selected from the group consisting of a sweetener, a bittering agent, and acidulant. A method for promoting expression of PGC-1α protein, or preventing conversion of a slow twitch muscle into a fast twitch muscle, the method including: administering Enterococcus faecium R30 strain (NITE BP-01362) or a processed product thereof to a subject in need thereof.

### CROSS-REFERENCE TO RELATED APPLICATIONS

The present application is a continuation of International Application No. PCT/JP2018/017661, filed on May 7, 2018, which is based on and claims the benefit of priority to Japanese Application No. 2017-092263, filed on May 8, 2017. The entire contents of these applications are incorporated herein by reference.

### BACKGROUND OF THE INVENTION

### Technical Field

The present invention relates to safe drugs and methods for promoting a protein expression of PGC-1α involved in energy production or for inhibiting slow-to-fast muscle conversion caused by lack of exercise or the like.

### Discussion of the Background

Muscle deterioration is problem in the rehabilitation of a hospitalized patient or the like, since muscle deterioration inhibits the elderly's independent activities of daily living (ADL). Thus, measures against muscle loss due to lack of exercise, sarcopenia and disuse muscle atrophy are required. Sarcopenia means muscle loss due to aging or disease. Disuse muscle atrophy means muscle atrophy caused by not using muscle for a long time due to bed rest or the like.

Muscle is roughly classified into two types, i.e. slow twitch muscle and fast twitch muscle. Slow twitch muscle (type I myofiber) is used for maintaining posture, endurance and the like. Fast twitch muscle (type II myofiber) momentarily exerts a large force. An improvement of a muscle mass and a muscle strength is desired for any types of muscle, but it is known that when slow twitch muscle, which is mainly used in daily life operations, is not used, slow twitch muscle becomes fast twitch muscle and decreases. In particular, in the case of elderly people and subjects with limited exercise, it is desirable not only to maintain the total muscle mass, but also to maintain muscle quality by preventing slow twitch muscle from becoming fast twitch muscle. So far, weight loss suppression of gastrocnemius muscle by branched chain amino acid (Non-patent document 1), prevention of fast twitch muscle formation accompanying disuse muscle atrophy by nucleoprotein (Non-patent document 2), slow twitch muscle formation from fast twitch muscle by high molecular weight polyphenol derived from fermented tea (Patent document 1), and the prevention of slow twitch muscle from becoming fast twitch muscle by a fruit polyphenol (Patent document 2) have been reported.

Lactic acid bacteria have attracted attention as an active ingredient which exerts beneficial physiological activity in a human and an animal with the background of health orientation in recent years. So far, various physiological activities have been known such as intestinal regulation action, antiallergic action, cholesterol reduction action, blood pressure lowering action by oral administration to a human and an animal (Non-patent document 3), and Atrogin-1 expression suppression action and muscle degradation suppression by lactic acid bacteria are reported by Patent document 3 as the action of lactic acid bacteria on muscle. The applicant of the present application has reported Enterococcus faecium as a lactic acid bacteria having an effect to recover from exhaustion and to improve blood flow (Patent Document 4).

### PATENT DOCUMENTS

Patent document 1: JP 2010-037323 A
Patent document 2: JP 2006-328031 A
Patent document 3: JP 2016-216408 A
Patent document 4: WO 2014/021205

### Non-Patent Documents

Non-patent document 1: Shimizu, N., et al., Cell Metabolism, 2011, 13, 170
Non-patent document 2: INAMOTO Kenta, et al., Physical Therapy Japan, vol. 39, Suppl. No. 2, Ab1109 (2012) Non-patent document 3: Fuller, R., J. Appl. Bacteriol., 1989, 66, 365

As mentioned above, although the component which has a function to prevent slow twitch muscle from becoming fast twitch muscle is discovered, there existed problems, such as acquisition of a raw material and troublesome purification from a raw material. On the one hand, lactic acid bacteria can be easily increased by cultivation. For example, the lactic acid bacteria described in Patent document 3 are expected to maintain a muscle mass, since the lactic acid bacteria have the action to suppress a decomposition of muscle; however, lactic acid bacteria which can maintain or improve a quality of muscle by a function to prevent slow twitch muscle from becoming fast twitch muscle have not been reported.

Under the above-described circumstances, the objective of the present invention is to provide safe drugs and methods which can promote a protein expression of PGC-1α involved in energy production or which can inhibit slow-to-fast muscle conversion caused by lack of exercise or the like.

The present inventors have intensively studied to solve the above problems. As a result, the inventors completed the present invention by finding that the specific bacterium belonging to enterococcus, which is a safe lactic acid bacterium, exhibits a function to promote PGC-1α protein expression and prevent slow twitch muscle from becoming fast twitch muscle.

### SUMMARY OF THE INVENTION

A first aspect of the present invention is directed to a food or a drink, including Enterococcus faecium R30 strain (NITE BP-01362) or a processed product thereof. The food or the drink may further include a sweetener, a bittering agent, acidulant, or a combination thereof.

A second aspect of the present invention is directed to a method for promoting expression of PGC-1α protein, including administering Enterococcus faecium R30 strain (NITE BP-01362) or a processed product thereof to a subject in need thereof.

A third aspect of the present invention is directed to a method for preventing conversion of a slow twitch muscle into a fast twitch muscle, including administering Enterococcus faecium R30 strain (NITE BP-01362) or a processed product thereof to a subject in need thereof.

The present invention further includes the following aspects:

[1] A PGC-1α protein expression promoter comprising Enterococcus faecium R30 strain (NITE BP-01362) as an active ingredient.

[2] The PGC-1α protein expression promoter according to the above [1], wherein the R30 strain is dead.

[3] The PGC-1α protein expression promoter according to the above [1] or [2], wherein the PGC-1α protein expression promoter is orally administered.

[4] A slow-to-fast muscle conversion inhibitor, comprising Enterococcus faecium R30 strain (NITE BP-01362) as an active ingredient.

[5] The slow-to-fast muscle conversion inhibitor according to the above [4], wherein the R30 strain is dead.

[6] The slow-to-fast muscle conversion inhibitor according to the above [4] or [5], wherein the slow-to-fast muscle conversion inhibitor is orally administered.

[7] A food and drink, comprising the PGC-1α protein expression promoter according to any one of the above [1] to [3] or the slow-to-fast muscle conversion inhibitor according to any one of the above [4] to [6].

[8] The food and drink according to the above [7], further comprising a package, wherein a function to prevent slow twitch muscle from becoming fast twitch muscle or a function pertaining thereto is displayed on the package.

[9] A method for promoting protein expression of PGC-1a, comprising a step of administering Enterococcus faecium R30 strain (NITE BP-01362) or processed Enterococcus faecium R30 strain (NITE BP-01362) to a subject.

[10] The method according to the above [9], wherein the R30 strain is dead.

[11] The method according to the above [9] or [10], wherein the R30 strain is orally administered.

[12] The method according to any one of the above [9] to [11], wherein slow twitch muscle is prevented from becoming fast twitch muscle.

[13] A method for preventing slow twitch muscle from becoming fast twitch muscle, comprising a step of administering Enterococcus faecium R30 strain (NITE BP-01362) or processed Enterococcus faecium R30 strain (NITE BP-01362) to a subject.

[14] The method according to the above [13], wherein the R30 strain is dead.

[15] The method according to the above [13] or [14], wherein the R30 strain is orally administered.

### BRIEF DESCRIPTION OF THE DRAWINGS

FIG. 1 is a graph showing the effect to promote PGC-1α protein expression by administering Enterococcus faecium R30 strain according to one embodiment of the present invention to a disuse muscle atrophy model rat.

FIG. 2 is a graph showing the effect to prevent slow twitch muscle from becoming fast twitch muscle by administering Enterococcus faecium R30 strain according to one embodiment of the present invention to a disuse muscle atrophy model rat.

FIG. 3 is a graph showing the effect to promote an oxidative metabolizing enzyme activity by administering Enterococcus faecium R30 strain according to one embodiment of the present invention to a disuse muscle atrophy model rat.

### DESCRIPTION OF THE EMBODIMENTS

The Enterococcus faecium R30 strain used in the present invention is a beneficial bacterium among lactic acid bacteria, and is classified into Enterococcus faecium species. In addition, Enterococcus is safe, since Enterococcus have been used in food for many years and is also present in the intestine. Furthermore, the Enterococcus faecium R30 strain used in the present invention exhibits an excellent functions to promote PGC-1a protein expression and prevent slow twitch muscle from becoming fast twitch muscle. Moreover, R30 strain can be produced in a large scale by cultivation. The present invention is, therefore, very useful, since the present invention can maintain a quality of muscle by safely suppressing a decrease of slow twitch muscle in a patient and elderly people of which slow twitch muscle necessary for daily life is decreased due to insufficient exercise caused by disease, injury, aging or the like.

The PGC-1α protein expression promoter and the slow-to-fast muscle conversion inhibitor according to embodiments of the present invention are characterized in including Enterococcus faecium R30 strain as an active ingredient. Hereinafter, Enterococcus faecium R30 strain is abbreviated as “R30 strain” in some cases.

Enterococcus faecium R30 strain was deposited with a depositary institution as follows:

(i) Name and address of depositary institution

Name: National Institute of Technology and Evaluation, NITE Patent Microorganisms Depositary

Address: #122, 2-5-8 Kazusakamatari, Kisarazu-shi, Chiba, Japan

(ii) Date of the original deposit: May 16, 2012

(iii) Date of receipt of request for conversion: Apr. 24, 2013

(iv) Accession number: NITE BP-01362

Microbiological characteristics of Enterococcus faecium R30 strain are described as follows.

TABLE 1

| Test item | Result | Test item | Result |
|---|---|---|---|
| Gram stainability | Positive | Sugar | D-Ribose+ |
| Catalase | Negative | assimilating | D-Mannitol+ |
| Spore | None | property | Lactose+ |
| Motility | None | | D-Sorbitol− |
| Degradation of | Positive | | D-Trehalose− |
| sodium pyruvate | | | D-Raffinose− |
| Degradation of | Positive | | |
| aesculin | | | |
| Degradation of | Positive | | |
| pyrrolidonyl-2- | | | |
| naphthylamide | | | |
| Degradation of | Positive | | |
| 2-naphthyl-β-D- | | | |
| galactopyranoside | | | |
| Degradation of | Positive | | |
| L-leucine-2- | | | |
| naphthylamide | | | |
| Degradation of | Positive | | |
| L-alginic acid | | | |

A method for cultivating R30 strain used in the present invention is not particularly restricted as long as R30 strain can be effectively cultivated by the method. For example, R30 strain can be cultivated by test tube cultivation, flask cultivation, cultivation in a fermenter, or the like. A culture medium is also particularly not restricted, and any culture media can be used as long as R30 strain can be effectively cultivated in the culture media. For example, MRS culture medium, which is generally used for cultivating lactic acid bacteria, can be used.

R30 strain used in the present invention may be alive or dead. The live bacterium means live R30 strain and contains not only proliferative R30 strain but also R30 strain which is not propagative in a dormant state but which becomes able to be propagative again in the presence of water or the like.

The dead bacterium means a bacterium killed by high temperature, high pressure, drug treatment or the like. A bacterium-killing treatment is not particularly restricted and exemplified by dry heat sterilization, steam sterilization, high pressure steam sterilization, chemical sterilization, ultrasonic wave sterilization, electromagnetic wave sterilization and ultraviolet ray sterilization. A bacterium-killing treatment may be performed after drying a collected bacterial body. A dead bacterium may be a dead bacterium body itself or a processed dead bacterium. An example of the processed dead bacterium includes a dead bacterium which is subjected to at least one treatment selected from grinding, fracturing, concentration, pasting, drying and diluting of the bacterium. A means to dry the bacterium is not particularly restricted and exemplified by spray dry, freeze dry and drum dry.

A dosage form of the PGC-1α protein expression promoter and the slow-to-fast muscle conversion inhibitor according to embodiments of the present invention is not particularly restricted. For example, a dried formulation or a dispersion itself of dead or live R30 strain may be used, it is preferred to prepare an easily administered dosage form. For example, embodiments of the present invention directed to a formulation may be used as an external preparation, since the formulation acts on muscle; however, it is preferred that the formulation may be used as an oral preparation, since the formulation shows a beneficial effect by being orally administered as Example described later and an oral preparation can be easily administered. Hereinafter, “the PGC-1α protein expression promoter and the slow-to-fast muscle conversion inhibitor” according to embodiments of the present invention are simply abbreviated as the “formulation”.

The dosage form is not particularly restricted and exemplified by tablet, powder, capsule, sugar-coated tablet, granule, extract, elixir, syrup, tincture and limonade. A pharmaceutically acceptable additive may be used in embodiments of the present invention directed to a formulation depending on the dosage form. An example of such an additive includes excipient, disintegrant, lubricant, antioxidant, fragrance, seasoning, sweetener, colorant, thickening stabilizer, color former, bleach, fungicide, gum base, bittering agent, enzyme, brightening agent, acidulant, emulsifier, toughening agent, manufacturing agent, binder, tonicity agent, buffering agent, solubilizing agent, preservative, stabilizer, dispersing agent, absorption enhancer and coagulant. An example of an excipient includes gum arabic, lactose, microcrystalline cellulose, starch, sodium starch glycolate, calcium hydrogen phosphate, magnesium stearate, talc and colloidal silicon dioxide. The formulation of one embodiment of the present invention can be produced by an ordinary method depending on the dosage form or the like.

For example, the PGC-1α protein expression promoter and the slow-to-fast muscle conversion inhibitor which contain live R30 strain can be obtained by collecting the R30 body cultivated by an ordinary method from a culture medium, adding a solution of a protectant to the collected bacterial body, drying the mixture, and mixing the dried mixture with an appropriate excipient.

The above-described protectant is a component which protects a bacterial body from an external environment, which maintains a live bacteria number or which inhibits a decrease of a live bacteria number. Such a protectant may be a substance to reduce a damage to a bacterial body, such as trehalose, bovine serum albumin, fat-free milk, sodium glutamate, L-ascorbic acid, histidine, malic acid, whey, glucose, aspartic acid, methionine, starch, dextrin, sucrose, lactose, sodium chloride and phosphate salt. One of the protectant may be used alone, or a combination of the protectants may be used. A content ratio of the protectant is not restricted, and the lower limit of a total amount of the protectant to a weight of a dried bacterial body is preferably 1 mass % or more. When the content ratio is 1 mass % or more, a damage to a bacterial body can be reduced more surely. On the one hand, the upper limit of the content ratio is not particularly restricted, and the content ratio may be adjusted to 50000 mass % or less. The content ratio is more preferably 10 mass % or more, even more preferably 50 mass % or more, even more preferably 100 mass % or more, and more preferably 20000 mass % or less, even more preferably 10000 mass % or less.

The formulation containing dead R30 strain can be obtained by, for example, collecting the R30 body cultivated by an ordinary method from a culture medium, killing the collected bacterial body, drying the killed bacterial body, and mixing the dried bacterial body with an appropriate excipient.

As Examples described later, when the formulation of one embodiment of the present invention is orally administered, the formulation exhibits the excellent effects to promote the protein expression of PGC-1α and prevent slow twitch muscle from becoming fast twitch muscle. The PGC-1α protein expression promoter and slow-to-fast muscle conversion inhibitor according to embodiments of the present invention, therefore, can be used as, for example, a food and drink product, a functional food, a supplement, a medicine, a quasi-drug, an animal feed, a pet food, a drug for animal, or the like.

The formulation of one embodiment of the present invention can be added into a general food and drink product. When the formulation is routinely ingested as a food and drink, a form of such a food and drink containing the formulation is not particularly restricted and exemplified by a beverage such as milk beverage, soft drink, isotonic drink, energy drink, beauty care drink and liquid nutritional supplement; a sweet stuff such as chewing gum, chocolate, candy, jelly, cake, pie, cookie, Japanese-style confectionery, snack, fried confectionery, rice confectionery, biscuit and cracker; a frozen dessert such as ice cream and ice; a noodle such as wheat noodle, Chinese noodle, spaghetti and instant noodle; a paste product such as boiled fish paste, chikuwa (fish paste shaped in a long tube) and hanpen (a white, square-shaped fish paste mixed with rice and yam powder); a seasoning such as dressing, mayonnaise, basting and sauce; other processed product such as bread, ham, rice porridge, rice, soup, retort food, frozen food, edible oil and fat composition, cooking oil, sprayed oil, butter, margarine, shortening, whipped cream, concentrated milk, whitener, dressing, pickling liquid, bakery mix, fried food, processed meat product, tofu (bean curd) and konnyaku (jelly made from devil's-tongue starch); a general food form such as jam, fermented milk and can. The food and drink may be a fermented product prepared by fermenting a general food and drink with lactic acid bacteria according to one embodiment of the present invention.

The embodiment of the present invention directed to a formulation can be used as health food, supplement, functional food, food with function claims, dietary supplement, food for specified health use, food with nutrient function claims, care food, smiley care food, digestion or swallowing auxiliary, concentrated fluid food, food for patient and dietary supplement. The formulation can be also used for pet food, livestock feed or the like.

In the cases of food for specified health use, food with function claims and dietary supplement, the PGC-1α protein expression promoter and the slow-to-fast muscle conversion inhibitor according to embodiments of the present invention may be packed in a package, and the function to promote a protein expression of PGC-1α or prevent slow twitch muscle from becoming fast twitch muscle may be displayed on the package. Such a package is not particularly restricted and may be exemplified by box, container, packaging film and wrapping paper. An example of the function to be displayed on the package includes functions to promote a protein expression of PGC-1α and prevent slow twitch muscle from becoming fast twitch muscle, and a function related thereto, such as improvement and sustention of endurance athletic performance; suppression of decrease and degradation of muscle and muscular strength; sustention and improvement of amount and quality of muscle; improvement, sustention and support of motor function, walk function and posture; support for movement of elderly people in daily life; and expression described in this disclosure as a purpose and effect of a function to promote a protein expression of PGC-1α or prevent slow twitch muscle from becoming fast twitch muscle. Of course, a function of which expression is different from the above description but which is similar to the above functions may be displayed.

The PGC-1α protein expression promoter of one embodiment of the present invention has an excellent function to promote a protein expression of PGC-1a. PGC-1α is peroxisome proliferator-activated receptor γ coactivator 1α, is expressed mainly in skeletal muscle, and has a function to activate mitochondria. It is known that a protein expression amount of PGC-1α is increased by exercise. Since mitochondria produces ATP from fat and sugar, PGC-1α may increase energy production in vivo. In addition, since PGC-1α exists mainly in type I muscle fiber, which is slow twitch muscle, PGC-1α is a determinant of muscle fiber type, and improvement and sustention of endurance athletic performance can be expected by promoting protein expression of PGC-1α.

An effect to promote protein expression of PGC-1α can be evaluated by, for example, measuring a protein expression amount of PGC-1α in soleus muscle of a hind limb suspension model animal with a Western blotting method using an anti-PGC-1α antibody as Examples described later. When a protein expression amount of PGC-1α in soleus muscle of a rat to which a test substance is administered is larger than that of a rat to which the test substance is not administered, the test substance can be evaluated to have an effect to promote a protein expression amount of PGC-1α.

The slow-to-fast muscle conversion inhibitor of one embodiment of the present invention has an excellent function to prevent slow twitch muscle from becoming fast twitch muscle. The slow-to-fast muscle conversion inhibitor of one embodiment of the present invention is useful for continuing an independent activity, and maintaining, recovering and improving an activity of daily living (ADL) and quality of life (QOL). Since a slow twitch muscle plays an important role for postural maintenance and walk, the slow-to-fast muscle conversion inhibitor is useful for improving health in combination with rehabilitation and stretching exercise. It is also effective to combine the slow-to-fast muscle conversion inhibitor with an exercise requiring an endurance, such as jogging and marathon, for which a slow twitch muscle is mainly used.

A “slow twitch muscle” is referred to as red muscle or inner muscle in some cases, and has a slow contractile velocity but has good endurance strength. A slow twitch muscle is always used in daily life for maintenance of posture, walk or the like. On the one hand, a “fast twitch muscle” is referred to as white muscle, and has a fast contractile velocity and good instantaneous force. The kind of a muscle fiber is mainly classified into slow twitch muscle type I and fast twitch muscle type II. Slow twitch muscle type I has a large amount of mitochondria and produces energy by using oxygen mainly. Fast twitch muscle type II has a small amount of mitochondria and produces energy by glycolytic system mainly. A slow twitch muscle fiber and a fast twitch muscle fiber may be respectively referred to as a slow twitch muscle and a fast twitch muscle in some cases. Type I and type II muscle fibers are mixed in various ratios in muscle. A ratio of type I muscle fiber is large in a slow twitch muscle, and a ratio of type II muscle fiber is large in a fast twitch muscle. An example of a slow twitch muscle includes soleus muscle, and an example of a fast twitch muscle includes extensor digitorum longus muscle.

The term “muscular atrophy” means that a muscle becomes thin and causes muscle weakness. Muscular atrophy is classified into myogenic muscle atrophy caused by a disease of a muscle itself, neurogenic muscle atrophy caused by a failure of a motor neuron, and disuse muscle atrophy caused by disuse of muscle. Disuse muscle atrophy is likely to occur when activity is decreased due to aging or hospitalization caused by injury or disease.

A conversion of slow twitch muscle to fast twitch muscle is a qualitative change of muscle and means that a ratio of type I muscle fiber as a slow twitch muscle fiber is decreased and a ratio of type II muscle fiber as a fast twitch muscle fiber is increased in a muscle fiber composition of slow twitch muscle. A content ratio of type I muscle fiber and type II muscle fiber is referred to as a type relative proportion in some cases. Both of muscle fiber diameters of type I muscle fiber and type II muscle fiber is decreased in muscle atrophy. When muscle atrophy is caused by low activity, such as the case of disuse muscle atrophy, it is observed that type I muscle fiber is decreased more remarkably than type II muscle fiber and a ratio of type II muscle fiber is increased. Soleus muscle is a slow twitch muscle which plays an important role for postural maintenance and walk, and remarkably becomes thin in disuse muscle atrophy of elderly people. It is also observed in an animal experiment under a zero gravity environment in space that soleus muscle as slow twitch muscle remarkably becomes thin and is changed to fast twitch muscle.

An example of an animal model of disuse muscle atrophy includes a hind limb suspension model and a denervated model. Other muscle atrophy model is exemplified by a steroid muscle atrophy animal model prepared by dexamethasone, which is characterized in that a decrease of a cross-sectional area specific to a fast muscle fiber and a weight change of soleus muscle having many slow muscle fibers are not observed and a decrease of gastrocnemius muscle having many fast muscle fibers is observed.

The effect of the slow-to-fast muscle conversion inhibitor according to one embodiment of the present invention can be evaluated by, for example, preparing a transverse section of soleus muscle of a hind limb suspension model animal and calculating a ratio of a type I muscle fiber and a type II muscle fiber by ATPase stain, SDH (succinate dehydrogenase) stain or the like to distinguish a type I muscle fiber and a type II muscle fiber from each other as Examples described later. When a relative proportion of a type I muscle fiber in soleus muscle of an experimental animal to which a test substance is administered is larger than that of an experimental animal to which the test substance is not administered, the test substance can be evaluated to have an effect to prevent slow twitch muscle from becoming fast twitch muscle.

The formulation of one embodiment of the present invention exhibits an excellent function to promote an oxidative metabolizing enzyme activity. An oxidative metabolism means a process to obtain energy from a carbohydrate such as sugar by TCA cycle of mitochondria and electron transport system. An oxidative metabolizing enzyme means an enzyme related to an oxidative metabolism. A citrate synthetase (CS enzyme) and a succinate dehydrogenase (SDH enzyme) are particularly important enzymes among oxidative metabolizing enzymes. CS enzyme is a rate determining enzyme of TCA cycle, and SDH enzyme works in both of TCA cycle and complex II of electron transport system. A slow twitch muscle fiber (type I muscle fiber) has a larger amount of mitochondria in comparison with a fast twitch muscle fiber (type II muscle fiber) and produces energy depending on an oxidative metabolism.

The effect to promote an oxidative metabolizing enzyme activity of one embodiment of the present invention directed to a formulation can be evaluated by, for example, measuring an activity of a citrate synthetase or a succinate dehydrogenase in soleus muscle of a hind limb suspension model as Examples described later. When an activity of a citrate synthetase or a succinate dehydrogenase in soleus muscle of an experimental animal to which a test substance is administered is higher than that of an experimental animal to which the test substance is not administered, the test substance can be evaluated to have an effect to promote an oxidative metabolizing enzyme activity.

The present invention also relates to a method for promoting a protein expression of PGC-1α and a method for preventing slow twitch muscle from becoming fast twitch muscle, characterized in comprising a step to administer R30 strain or processed R30 strain to a subject. The subject is not particularly restricted as long as it is needed to prevent slow twitch muscle from becoming fast twitch muscle for the subject, and is preferably exemplified by elderly people and a person in need of nursing care with few opportunities to move muscles, and a patient and a person under a medical treatment for a long time who spend a long time in bed due to illness or injury. R30 strain may be administered in the form of the above-described formulation, i.e. the PGC-1α protein expression promoter and the slow-to-fast muscle conversion inhibitor of embodiments of the present invention.

A frequency and an amount of dose of the formulation of the embodiment of the present invention may be appropriately adjusted depending on the age, sex, condition or the like of the subject. A dose of R30 strain per one day may be appropriately adjusted and for example, may be 0.001 mg/kg body weight or more and 1000 mg/kg body weight or less. The dose is preferably 0.01 mg/kg body weight or more, more preferably 0.1 mg/kg body weight or more, and preferably 500 mg/kg body weight or less. The frequency of dose per one day may be also appropriately adjusted and may be 1 time or more and 5 times or less. The subject to be administered is not particularly restricted as long as it is needed to prevent slow twitch muscle from becoming fast twitch muscle for the subject as described above, and may be exemplified by a subject whose muscle mass and muscle strength are decreased or are expected to be decreased by a decrease of an activity amount due to illness, injury, aging or the like.

The formulation of one embodiment of the present invention can be administered to an animal except for a human in addition to a human. In other words, the present invention also relates to a method for preventing slow twitch muscle from becoming fast twitch muscle, characterized in comprising a step of administering the present invention formulation to an animal. An example of an animal except for a human includes a cultured animal, a companion animal and a race animal. A cultured animal is not particularly restricted, and is exemplified by a livestock such as horse, cattle, pig, sheep, goat, camel and llama; an experimental animal such as mouse, rat, guinea pig and rabbit; a poultry such as chicken, duck, turkey and ostrich; fishes; a crustacean; and a shellfish. A companion animal is not particularly restricted and is exemplified by dog and cat. A race animal is not particularly restricted and is exemplified by racehorse.

The formulation of one embodiment of the present invention can be appropriately combined with resistance exercise, physical therapy, rehabilitation, stretch, inner muscle training, or the like in order to improve the effect to increase or maintain a muscle mass and a muscle strength. The formulation is also combined with a daily exercise. An example of such a daily exercise includes not only stretching-out exercise, beauty salon exercise and avocational sport but also a light work such as a commute to and from work, a work and a housework.

The formulation of one embodiment of the present invention can be used in combination with other drug, a food having a function to intensify muscle, or the like in order to improve the effect to increase or maintain a muscle mass and a muscle strength. Such a food having a function to intensify muscle is not particularly restricted, and exemplified by a protein such as milk protein, whey protein, casein, soy protein, soy peptide, wheat protein, collagen and gelatin; a peptide such as whey peptide, casein peptide, casein phosphopeptide, wheat peptide and soy peptide; an amino acid such as valine, leucine, isoleucine, arginine, citrulline, ornithine, threonine, lysine, tryptophan, phenylalanine and methionine; a vitamin such as vitamin D and vitamin K; a component said to be effective for maintaining a joint and a bone, such as glucosamine, N-acetylglucosamine, hyaluronan, collagen, proteoglycan, methyl sulfonyl methane (MSM), calcium, magnesium and zinc; creatine; and β-hydroxy-β-methylbutanoic acid (HMB). In addition, the formulation can be combined with a food and a material having a function to combust body fat and control a body, such as onion, ginger, hot pepper, garlic, a gingko leaf, black ginger, licorice, and an extract thereof; a supplement material such as quercetin, polyphenol, capsaicin, carnitine and allicin; a unsaturated fatty acid such as ω-3 fatty acid; a microorganism such as lactic acid bacteria and yeast; seaweed; and dietary fiber. Other component may be a functional component for nutritional enhancement. An example of a functional component includes a vitamin, a trace metal, an amino acid, coenzyme Q10, an oligosaccharide, a dietary fiber, a chondroitin sulfate, fucoidan, fucoxanthin, astaxanthin, a catechin, a unsaturated fatty acid, a polyphenol, sesamin, placenta, yeast extract, black vinegar concentrate, garlic extract, ginkgo leaf extract, bilberry extract, blueberry extract, gensing extract, maca extract, bean seed coat extract, St. John's wort extract, pine bark extract, acai extract, noni extract, egg yolk extract, honey processed product and brown sugar lump. An example of an amino acid includes a branched chain amino acid and ornithine. Conzyme Q10 may be reduced or oxidized. An example of an unsaturated fatty acid includes ARA, EPA, DHA, α-linolenate, ω-linolenate and oleate. For example, a hydrophobic extract of licorice contains glabridin and has a function to increase a muscle mass. It is therefore possible to obtain a useful formulation by which not only a muscle mass can be increased or maintained but also a quality of muscle can be maintained or improved by combining a hydrophobic extract of licorice with R30 strain according to the present invention. In addition, R30 strain can be combined with useful lactic acid bacteria.

When the formulation of one embodiment of the present invention is used as a food and drink product, a content of R30 strain is not particularly restricted and may be appropriately adjusted. For example, a content of R30 strain in a food product may be 0.00001 mass or more and 50 mass % or less. The content is preferably 0.001 mass % or more, more preferably 0.01 mass % or more, and preferably 30 mass % or less.

The formulation of one embodiment of the present invention can be a food with health claims and a functional food in the form of a supplement, such as a capsule and a tablet. An example of a food with health claims includes a food for specified health use and a food with nutrient function claims, and an example of a functional food includes a food with function claims, a health food, a nutraceutical food and a dietary supplement. Since R30 strain also exhibits the functions such as prevention and improvement of fatigue; improvement of athletic ability and endurance; prevention and improvement of cold constitution, skin problem and back problem; reduction and alleviation of stool odor; growth promotion, it may be displayed that R30 strain is used for the functions and uses. With respect to a dose of a formulation of one embodiment of the present invention in such a case, the formulation may be administered in one time or several divided doses so that a dose of R30 strain per one day for an adult becomes 0.01 mg/kg body weight or more, preferably 0.1 mg/kg body weight or more, and 1000 mg/kg body weight or less, preferably 300 mg/kg body weight or less.

When the formulation of one embodiment of the present invention is used as a functional food or a supplement, a dosage form thereof is not particularly restricted and exemplified by capsule, syrup, tablet, ball, powder, granule, drinkable preparation, injection preparation, transfusion, collunarium, ophthalmic solution, suppository, adhesive skin patch and aerosolized agent. A pharmaceutically acceptable other component can be appropriately added for a preparation, such as excipient, disintegrant, lubricant, binder, antioxidant, colorant, aggregation preventing agent, absorption promoter, dissolution adjuvant and stabilizer.

When the formulation of one embodiment of the present invention is used as a pet food, a feed or a drug for an animal, the formulation may be used as a preparation containing R30 strain as an active ingredient or R30 strain may be appropriately added into a general feed depending on a kind of an animal, growth stage, and rearing environment such as region. An example of a raw material of a general feed includes grain, processed grain, chaff and bran, plant-derived oil cake, animal raw material, mineral, vitamin, amino acid, yeast such as beer yeast, and fine powder of inorganic substance. An example of grain or processed grain includes corn, milo, barley, wheat, rye, oat, millet, flour and wheat germ powder. An example of chaff and bran includes wheat bran, rice bran and corn gluten feed. An example of plant-derived oil cake includes soybean oil cake, sesame oil cake, cotton seed oil cake, peanut chaff, sunflower chaff and safflower chaff. An example of animal raw material includes fat-free milk, fish meal and meat-and-bone meal. An example of mineral includes calcium carbonate, calcium phosphate, sodium chloride and anhydrous silicic acid. An example of vitamin includes vitamin A, vitamin D, vitamin E, vitamin K, vitamin B1, vitamin B2, vitamin B6, vitamin B12, calcium pantothenate, nicotinamide and folic acid. An example of amino acid includes glycine and methionine. An example of fine powder of inorganic substance includes crystalline cellulose, talc, silica, white mica and zeolite.

The feed of one embodiment of the present invention may contain an additive for feed, which is generally used in a mixed feed, and other component as requested in addition to the above-described feed raw material. An example of such an additive for feed includes excipient, extender, binder, thickener, emulsifier, colorant, fragrance, food additive and seasoning. An example of other component except for an additive for feed includes antibiotic, germicide, insectifuge and preservative.

A dosage form of the feed according to one embodiment of the present invention is not particularly restricted, and is exemplified by powder, granule, paste, pellet, capsule and tablet. A capsule may be either of a hard capsule or a soft capsule. An animal to which the feed of one embodiment of the present invention is fed is not particularly restricted, and is exemplified by a livestock such as cattle, horse, pig and sheep; a domestic poultry such as chicken, turkey and domestic duck; an experimental animal such as mouse, rat and guinea pig; a pet such as dog and cat. A chicken includes both of broiler and hen.

### EXAMPLES

Hereinafter, embodiments of the present invention are described in more detail with Examples. The present invention is, however, not restricted to the following Examples in any way, and it is possible to work the present invention according to the Examples with an additional appropriate change within the range of the above descriptions and the following descriptions. Such a changed embodiment is also included in the technical scope of the present invention.

### Example 1: Preparation of Freeze-Dried Bacterial Body of Enterococcus faecium R30 Strain

MRS bouillon manufactured by KANTO CHEMICAL CO., INC. (0.52 g) was dissolved in water (10 mL). Then, the solution was sterilized by heat in an autoclave at 121° C. for 15 minutes to prepare MRS liquid culture medium. A glycerol stock suspension of Enterococcus faecium R30 strain (100 μL) was inoculated in the culture medium (10 mL), and R30 strain was cultivated at 37° C. for 24 hours to obtain a preliminary cultivated liquid. The preliminary cultivated liquid (1 mL) was added to the above MRS liquid culture medium (100 mL), and R30 strain was further cultivated at 37° C. for 24 hours. The mixture was subjected to centrifugation at 8000 rpm for 10 minutes after the cultivation to separate the culture medium and the bacterial body. The bacterial body was washed with sterilized water (100 mL) and then subjected to centrifugation again to separate the sterilized water and the bacterial body. Sterilized water (2 mL) was added to the bacterial body. The mixture was sterilized by heat at 90° C. for 30 minutes and freeze-dried to obtain freeze-dried bacterial body of Enterococcus faecium R30 strain.

### Example 2: Confirmation Test of Function to Promote Protein Expression of PGC-1α by Enterococcus faecium R30 Strain

Fifteen male SD rats of 11-week age were arbitrarily divided into 3 groups of control group (CON group), hind limb unloaded group (HU group) and hind limb unloaded+Enterococcus faecium R30 strain administration group (HU+R30 group). Each group had five rats. Each group was preliminarily raised for 1 week with free intake of water in addition to a general feed (“CE-2” manufactured by CLEA Japan). Then, the freeze-dried bacterial body of Enterococcus faecium R30 strain prepared in the above-described Example 1 was dispersed in saline, and the dispersion was orally administered to HU+R30 group one time per one day by using a sonde so that the dosage amount of the bacterial body became 500 mg per 1 kg of body weight. The same amount of saline was orally administered to CON group and HU group one time per one day so that the groups were subjected to a similar stress. Further, HU group and HU+R30 group were subjected to tail suspension treatment by using modified Morey method to induce disuse muscular atrophy by maintaining hind limbs in an unloaded condition. During the hind limb unloading period, the rats were able to move within the breeding cage by using the forelimb and allowed to ingest a general feed and water similarly to the preliminary breeding. After raising each group rat for two weeks, the soleus muscle of the hind limbs was taken out under anesthesia by pentobarbital and rapidly frozen by using liquid nitrogen.

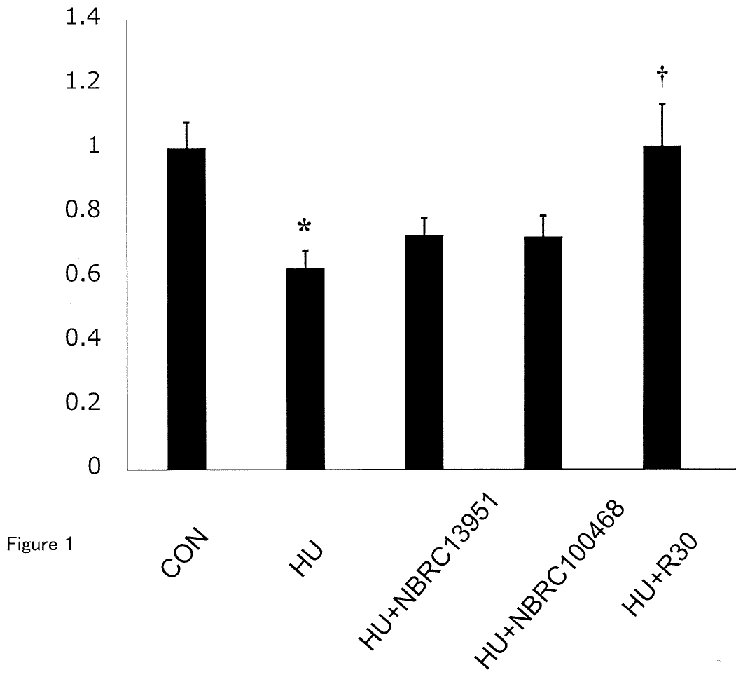

The soleus muscle frozen for preservation was homogenized in HEPES buffer additionally containing a protease inhibitor, and then the mixture was subjected to centrifugation to obtain the supernatant. The total mass of proteins in the supernatant was measured, and the concentrations of the samples were adjusted so that the total masses of proteins were the same between the samples and the sample was subjected to SDS-PAGE. The proteins developed by electrophoresis were transferred on a PVDF membrane, and an anti-PGC-1α antibody (“sc-13067” manufactured by Santa Cruz Biotechnology) solution diluted by 200 times or an anti-GAPDH antibody (“Y3322GAPDH” manufactured by Biochain Institute) solution diluted by 10000 times was added thereto after blocking to be incubated. Then, a secondary antibody labeled by HRP was further added thereto, and the membrane was colored by substrate for HRP detection (“EzWestLumi One” manufactured by ATTO). The image was fetched by using a luminescent image analyzer (“LAS-1000” manufactured by FUJIFILM Corporation), and an optical density was measured by using an image analysis software (“Science Lab” manufactured by FUJIFILM Corporation) to determine a protein expression amount of PGC-1α. One-way analysis of variance and Turkey-Kramer multiple comparison test were performed on the measured values. The result is shown in FIG. 1. In FIG. 1, “*” indicates a significant difference to the CON group at p<0.05, and “†” indicates a significant difference to the HU group at p<0.05.

As the result shown in FIG. 1, a protein expression amount of PGC-1α was significantly decreased in the HU group in comparison with the CON group. The reason may be that a load was not applied on the hind limb in the HU group. On the one hand, it was found that PGC-1α was biosynthesized in the HU+R30 group, to which Enterococcus faecium R30 strain prepared in Example 1 was orally administered, similarly to the CON group, to which a load was applied on the hind limbs under normal conditions.

### Example 3: Confirmation Test of Function to Prevent Slow Twitch Muscle from Becoming Fast Twitch Muscle

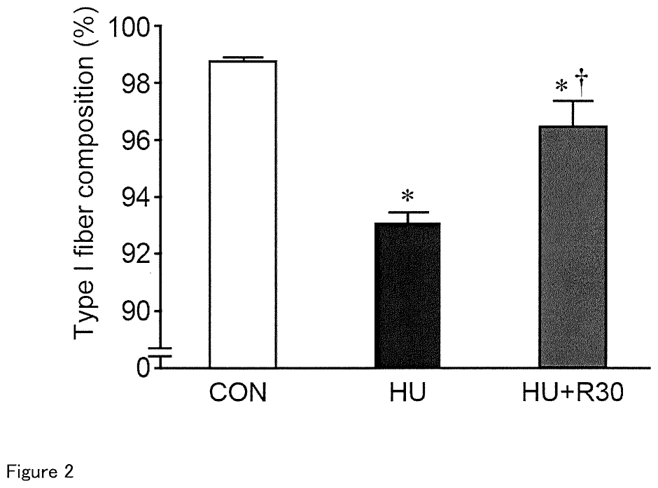

Frozen sections having a thickness of 12 μm were prepared from the soleus muscle samples obtained in the above-described Example 2 by using a cryostat and attached on glass slides. After ATPase was selectively deactivated by using a barbital solution of pH 4.3, type I fiber was stained. The stained section was observed by using an optical microscope to calculate a ratio of type I fiber to a total muscle fiber. The result is shown in FIG. 2. In FIG. 2, “*” indicates a significant difference to the CON group at p<0.05, and “†” indicates a significant difference to the HU group at p<0.05.

As the result shown in FIG. 2, the ratio of type I fiber corresponding to slow twitch muscle was significantly decreased and fast twitch muscle progressively changed to slow twitch muscle in the HU group, of which hind limb was not loaded. On the one hand, a ratio of type I fiber was significantly decreased in the HU+R30 group in comparison with the CON group, but a ratio of type I fiber was significantly improved in comparison with the HU group. It was experimentally demonstrated from the above results that slow twitch muscle progressively changes to fast twitch muscle due to disuse muscle atrophy but the transition of slow twitch muscle to fast twitch muscle can be significantly declined by orally administering Enterococcus faecium R30 strain used in the present invention.

### Example 4: Confirmation Test of Function to Improve Oxidative Metabolizing Enzyme Activity

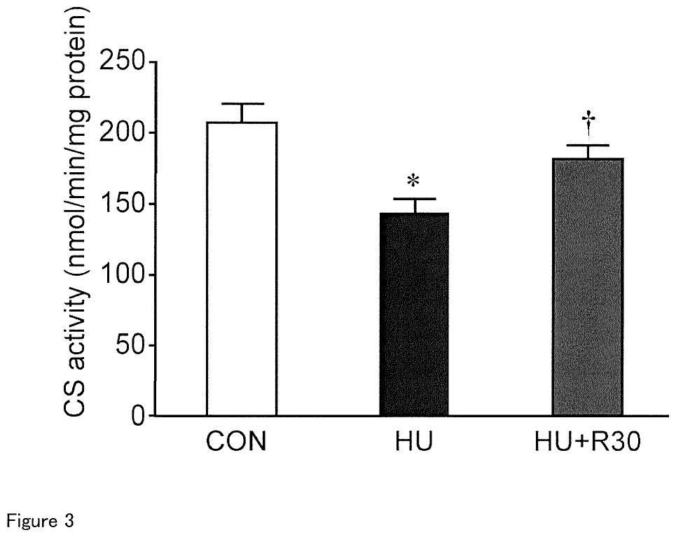

The supernatant was obtained from the homogenate of a soleus muscle sample similarly to the above-described Example 2 as an enzyme liquid. A mixed liquid of DTNB and acetyl-CoA was added to the enzyme liquid, and oxaloacetic acid was added thereto so that the final concentration became 0.5 mM to start the reaction. An absorbance was measured at 412 nm every 2 minutes over 8 minutes from the start of the reaction to determine the activity of citrate synthase as an oxidative metabolizing enzyme in the soleus muscle sample. The total protein mass was determined by Bradford method, and the measured reaction rate was standardized by using the value of the total protein mass. The result is shown in FIG. 3. In FIG. 2, “*” indicates a significant difference to the CON group at p<0.05, “†” indicates a significant difference to the HU group at p<0.05, and “CS” is citrate synthase.

As the result shown in FIG. 3, the activity of citrate synthase, which is an important enzyme in TCA cycle involved in energy production, was significantly decreased and the energy metabolism was depressed in the unloaded hind limb of the HU group. On the one hand, it was experimentally demonstrated that the activity of citrate synthase was significantly improved and the energy metabolism was maintained in the HU+R30 group, though the HU+R30 group was not similarly loaded.

### Comparative Example 1: Preparation of Frozen Dried Bacterial Body of Lactobacillus acidophilus NBRC13951 Strain

Frozen dried bacterial body of Lactobacillus acidophilus NBRC13951 strain was prepared similarly to Example 1 except that Lactobacillus acidophilus NBRC13951 strain was used instead of Enterococcus faecium R30.

### Comparative Example 2: Preparation of Frozen Dried Bacterial Body of Enterococcus faecium NBRC100486 Strain

Frozen dried bacterial body of Enterococcus faecium NBRC100486 strain was prepared similarly to Example 1 except that Enterococcus faecium NBRC100486 strain was used instead of Enterococcus faecium R30.

### Comparative Example 3: Confirmation Test of Function to Promote Protein Expression of PGC-1α by Lactobacillus acidophilusNBRC13951 Strain and Enterococcus faecium NBRC100486 Strain

A function to promote protein expression of PGC-1α was evaluated similarly to the above-described Example 2 except that six SD rats were used and Lactobacillus acidophilus NBRC13951 strain prepared by the above-described Comparative example 1 or Enterococcus faecium NBRC100486 strain prepared by the above-described Comparative example 2 was used instead of Enterococcus faecium R30 strain. The result is shown in FIG. 1.

As FIG. 1, it was confirmed that a function to promote protein expression of PGC-1α is specific to R30 strain, since a protein expression amount of PGC-1α was decreased in the HU+NBRC13951 group which ingested NBRC13951 strain and the HU+NBRC100486 group which ingested NBRC100486 strain similarly to the HU group, though the bacterial body was orally administered in the same amount as the HU+R30 group.

### Comparative Example 4: Confirmation Test of Function to Prevent Slow Twitch Muscle from Becoming Fast Twitch Muscle by Lactobacillus Acidophilus NBRC13951 Strain and Enterococcus faecium NBRC100486 Strain

Frozen sections were prepared from the soleus muscle sample prepared by the above-described Comparative example 3 similarly to the method of Example 3, and the stained sections were observed by using an optical microscope.

As a result, a ratio of type I fiber was decreased in the HU+NBRC13951 group which ingested NBRC13951 strain and the HU+NBRC100486 group which ingested NBRC100486 strain in comparison with the HU+R30 group, though lactobacillus was orally administered in the same amount. It was confirmed from the result that a function to prevent slow twitch muscle from becoming fast twitch muscle is specific to R30 strain.

1. A food or a drink, comprising:
Enterococcus faecium R30 strain (NITE BP-01362) or a processed product thereof; and
at least one additive selected from the group consisting of a sweetener, a bittering agent, and acidulant.

2. The food or the drink according to claim 1, further comprising:
a package containing the food or the drink,
wherein the package displays a function of the food or the drink to promote expression of PGC-1α or to prevent conversion of slow twitch muscle into fast twitch muscle.

3. A method for promoting expression of PGC-1α protein, comprising:
administering Enterococcus faecium R30 strain (NITE BP-01362) or a processed product thereof to a subject in need thereof.

4. The method of claim 3, wherein the processed product is obtained by grinding, fracturing, concentrating, pasting, spray drying, freeze drying, drum drying, or diluting the R30 strain.

5. The method of claim 3, wherein the R30 strain or the processed product is orally administered to the subject.

6. The method of claim 3, wherein 0.001 mg/kg body weight or more and 1000 mg/kg body weight or less of the R30 strain or the processed product is administered to the subject.

7. The method of claim 3, wherein 0.1 mg/kg body weight or more and 500 mg/kg body weight or less of the R30 strain or the processed product is administered to the subject.

8. The method of claim 3, wherein the subject is a human.

9. The method of claim 3, wherein the subject is undergoing resistance exercise, physical therapy, rehabilitation, stretch, inner muscle training, or a combination thereof.

10. A method for preventing conversion of a slow twitch muscle into a fast twitch muscle, comprising:
administering Enterococcus faecium R30 strain (NITE BP-01362) or a processed product thereof to a subject in need thereof.

11. The method of claim 10, wherein the processed product is obtained by grinding, fracturing, concentrating, pasting, spray drying, freeze drying, drum drying, or diluting the R30 strain.

12. The method of claim 10, wherein the R30 strain or the processed product is orally administered to the subject.

13. The method of claim 10, wherein 0.001 mg/kg body weight or more and 1000 mg/kg body weight or less of the R30 strain or the processed product is administered to the subject.

14. The method of claim 10, wherein 0.1 mg/kg body weight or more and 500 mg/kg body weight or less of the R30 strain or the processed product is administered to the subject.

15. The method of claim 10, wherein the subject is a human.

16. The method of claim 10, wherein the subject is undergoing resistance exercise, physical therapy, rehabilitation, stretch, inner muscle training, or a combination thereof.
